(19) United States
(12) Patent Application Publication
(10) Pub. No.: US 2019/0315669 A1
(43) Pub. Date: Oct. 17, 2019

(54) PROCESS FOR THE PREPARATION OF HU-910 AND CRYSTALLINE STRUCTURE THEREOF

(71) Applicant: Yissum Research Developement Company of the Hebrew University of Jerusalem Ltd., Jerusalem (IL)

(72) Inventors: Jean-Michel ADAM, Village-Neuf (FR); Pascal DOTT, Rixheim (FR); Marcus HOHLER, Zeiningen (CH); Tanja BURGER, Riehen (CH); Orazio TAGLIENTE, Muttenz (CH); Ernst MISCHLER, Obrwil (CH)

(73) Assignee: Yissum Research Development Company of The Hebrew University of Jerusalem Ltd., Jerusalem (IL)

(21) Appl. No.: 16/347,838

(22) PCT Filed: Nov. 13, 2017
(86) PCT No.: PCT/IL2017/051232

§ 371 Date: May 7, 2019

Related U.S. Application Data
(60) Provisional application No. 62/421,308, filed on Nov. 13, 2016.

(51) Int. Cl. C07C 33/34

(52) U.S. Cl. CPC C07C 33/34; C07B 2200/13

(57) ABSTRACT

The invention provides processes for the preparation of HU-910, which are scalable to industrial purposes, using safer reagents and having high yield and pure product and a crystalline structure of HU-910, which is a unique product thereof.

### TECHNOLOGICAL FIELD

The present invention provides a process for the preparation of HU-910, which is scalable to industrial purposes, using safer reagents and having high yield and pure product and a crystalline structure thereof.

### BACKGROUND

((1S,4R)-2-(2,6-dimethoxy-4-(2-methyloctan-2-yl)phenyl)-7,7-dimethylbicyclo[2.2.1]hept-2-en-1-yl)methanol (HU-910) is a CB(2) receptor agonist.

WO2011061744, US20070060636 describe the process for preparing compound HU-910 as presented in the Scheme 1 below:

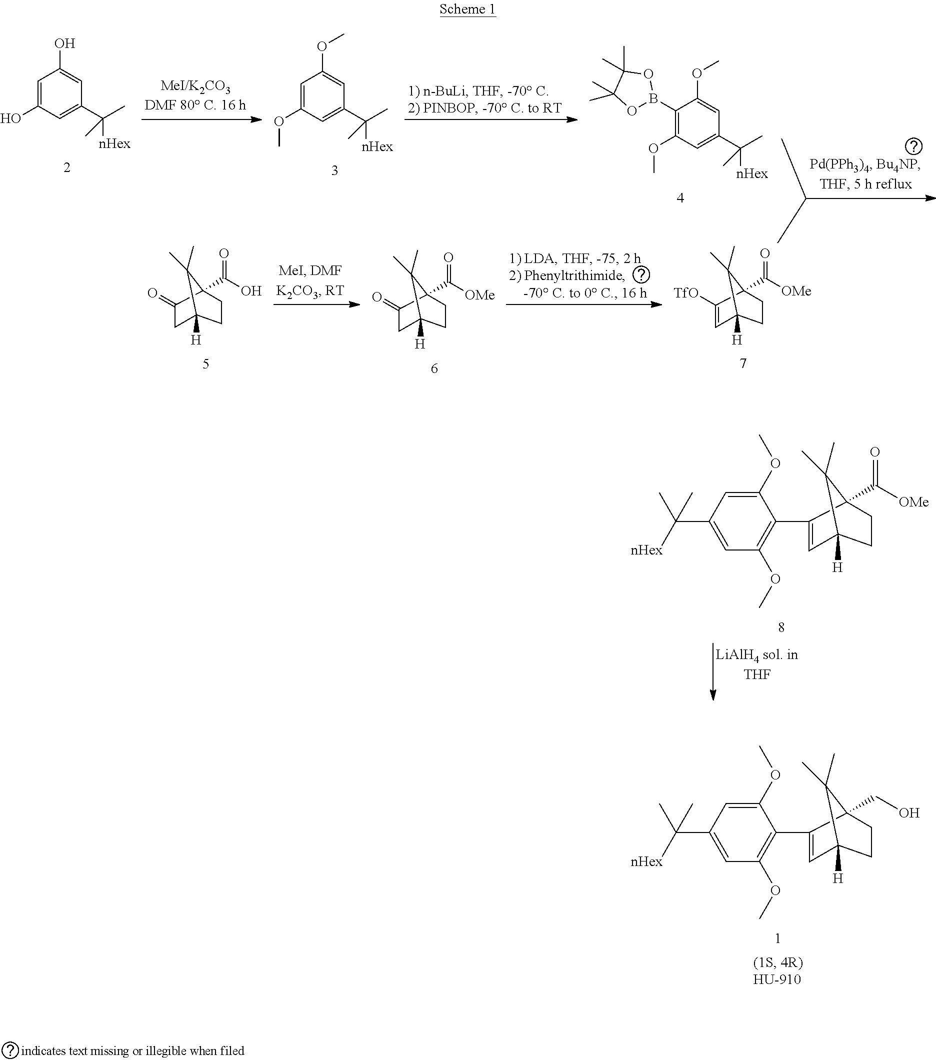

The synthesis in Scheme 1 relies on chromatography for purification due to the lack of solid intermediates. The present invention describes a route void of any chromatographic purification. The purity of HU-910 1 can be greatly impacted by the quality of the starting materials 2 (or 3). Commercial 2 or 3 may contain alkyl side chain analogs impurities. These impurities are very difficult to remove without preparative HPLC purification methods and are then typically found in the final product 1. Therefore, access to highly pure 2 or 3 is of great interest.

A new route to high purity building block 3 leading to high purity HU-910 is described in the present invention.

### SUMMARY OF THE INVENTION

The present invention provides a process for the preparation of HU-910 including the steps of:
(a) Preparation of resorcinol building block comprising:

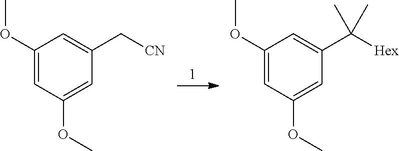

(b) Preparing the triflate ketopinic methyl ester derivative building block comprising:

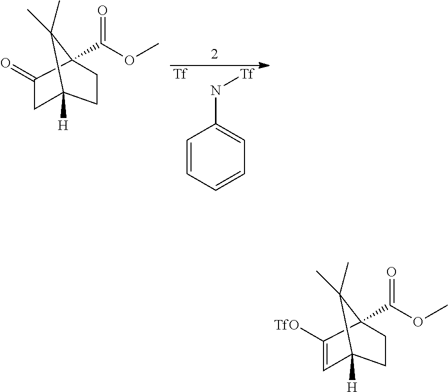

(c) Coupling the resorcinol building block and the triflate ketopinic methyl ester derivative building block comprising to produce a coupled compound:

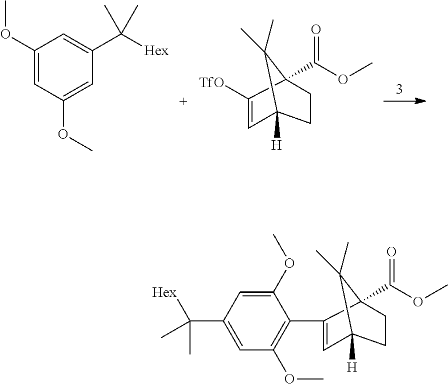

(d) Forming compound HU-910 from said coupled compound:

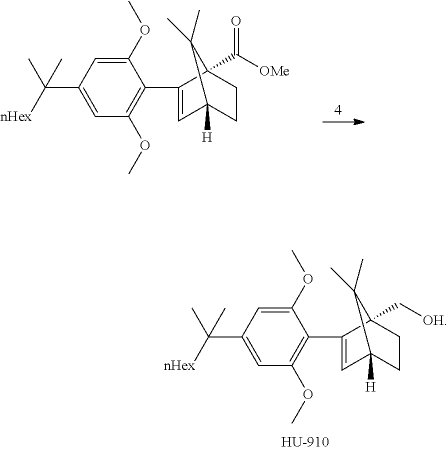

In some embodiments, step 1 of the process of the invention comprises the reactions: (1a) dimethylation reaction; (1b) a reduction reaction; (1c) an olefination reaction and (1d) a hydrogenation reaction. The reactions (1a)-(1d) are performed successively one after the other using the product of the previous reaction in the next one.

In some embodiments, said dimethylation reaction (1a) is performed in the presence of KOtBu and MeI in THF. In some embodiments reaction (1a) is:

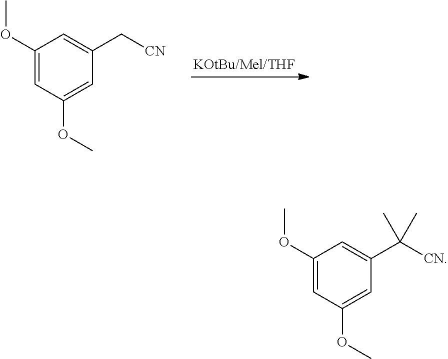

In some further embodiments, said reduction reaction (1b) is performed in the presence of DIBAH. In some embodiments, said reaction (1b) is:

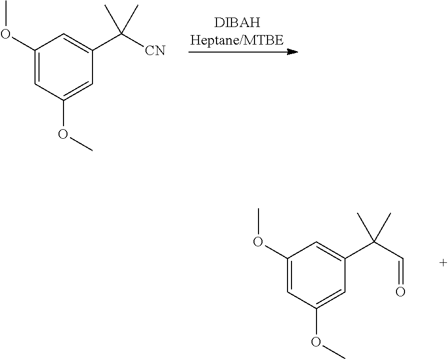

In other embodiments, said olefination reaction (1c) is a Wittig reaction. In some embodiment, said olefination is performed in the presence of a pentyl(triphenyl)phosphonium halide or pentylidene(triphenyl)-phosphane. In some embodiments, said olefination reaction (1c) is:

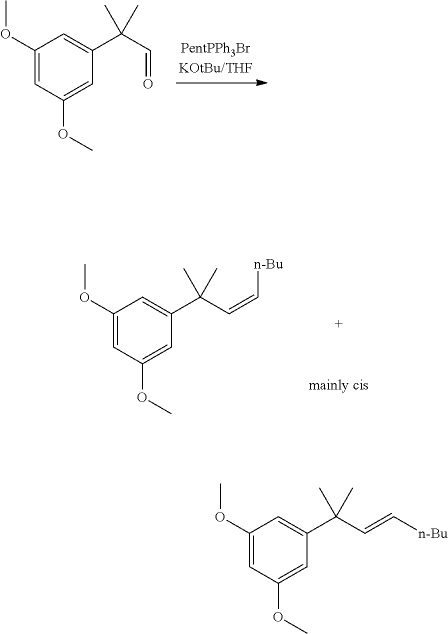

In other embodiments, said hydrogenation reaction (1d) is performed in the presence of a Pd catalyst. In some embodiments, step (1d) is:

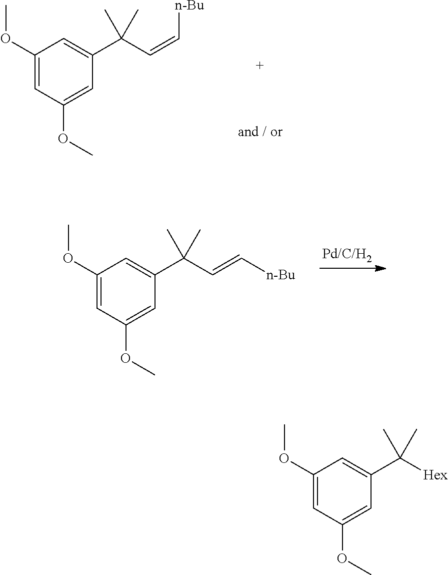

In some other embodiments, step 2 comprises the reactions: (2a) esterification reaction (2b) triflation reaction. The reactions (2a)-(2b) are performed successively one after the other using the product of the previous reaction in the next one.

In some embodiments, said esterification reaction (2a) is a Fischer esterification performed in the presence of an acid. In some embodiments said acid is gaseous HCl, concentrated HCl or concentrated H₂SO₄, preferably gaseous HCl. Gaseous HCl can alternatively be prepared by reaction of acetyl chloride with an alcohol. In some embodiments, step (2a) is:

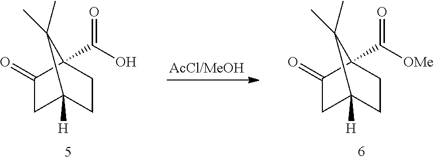

In other embodiments, said deprotonation reaction (2b) is performed in the presence of a base, in some embodiments said base is LDA. In some embodiments said base is selected from LDA, LiHMDS, KHMDS, NaHMDS, preferably LDA. The deprotonation is performed at a temperature of −80° C. to room temperature, preferably between −40° C. and 25° C., more preferably around 0° C. In some embodiments, step (2b) and the triflation is:

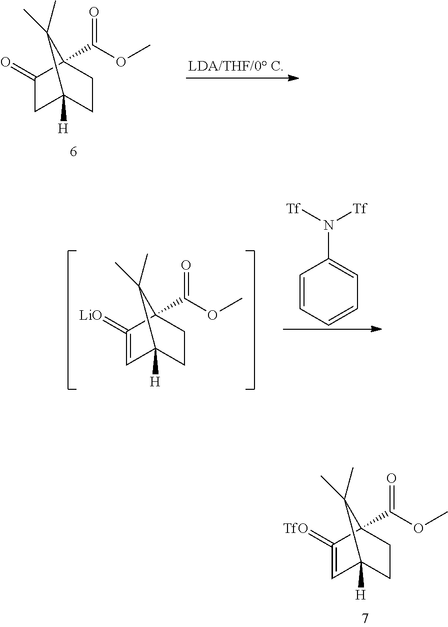

In some embodiments, step 3 comprises the reactions: (3a) a deprotonation of the dimethoxyaryl ring (3b) a reaction of the generated arylmetal with an electrophile (3c) a C—C coupling reaction. In some embodiments step (3c) is a Negishi coupling reaction. In further embodiments, step (3b) is performed with ZnBr₂.

In some embodiments step 3 performed by first deprotonating compound 3 with BuLi or s-BuLi, preferably BuLi. The deprotonation is performed at −60° C. to room temperature, preferably at −20° C. to room temperature, more preferably around 15° C. The resulting aryllithium intermediate 9 is transmetalled with a zinc halide, preferably zinc chloride or zinc bromide, more preferably zinc bromide, to generate an aryl zinc intermediate 10. Compound 10 can undergo a Negishi coupling reaction with triflate 7. The coupling reaction is performed in the presence of a Pd catalyst like (but not limited to) Pd(PPh)₃Cl₂ or Pd(PPh₃)₄. In some embodiments, said Negishi coupling is:

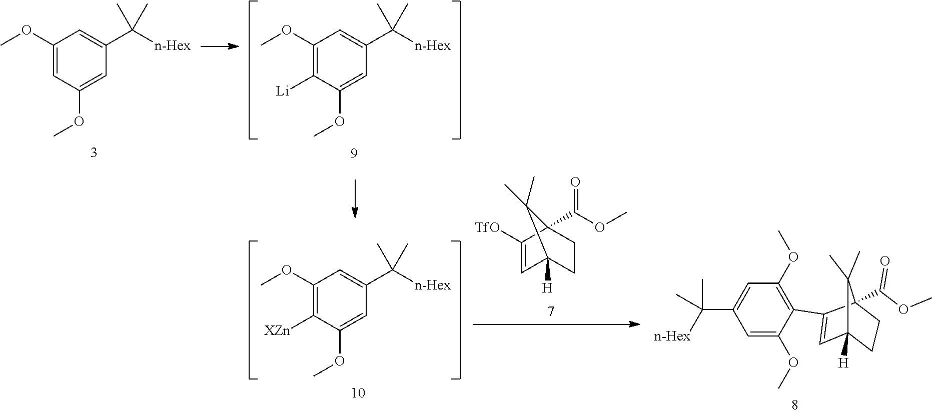

In some embodiments, step 4 comprises a hydrolysis step. In further embodiments, step 4 comprises the reactions: (4a) a hydrolysis reaction (4b) an acid reduction. In yet further embodiments, step 4 comprises the reactions: (4a) a hydrolysis (4b) an acid chloride formation and (4c) a reduction.

In some embodiments, said hydrolysis step in step 4 is performed in the presence of KOtBu, water and MeTHF and small amount of water. In some embodiments step (4a) is:

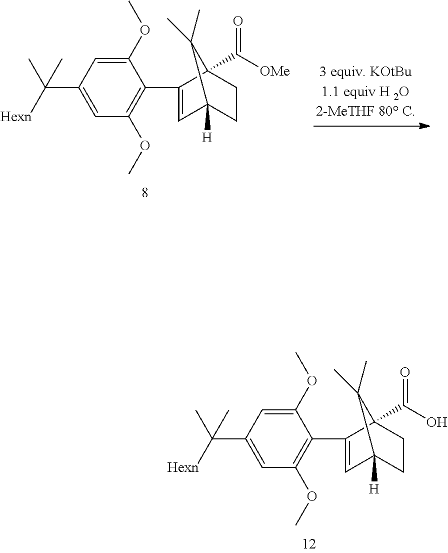

In some embodiments, said acid reduction reaction (4b) is performed by first transforming the acid 12 to the corresponding acid chloride then performing the reduction. Alternatively, the acid 12 can be directly reduced. In some embodiments step (4b) is:

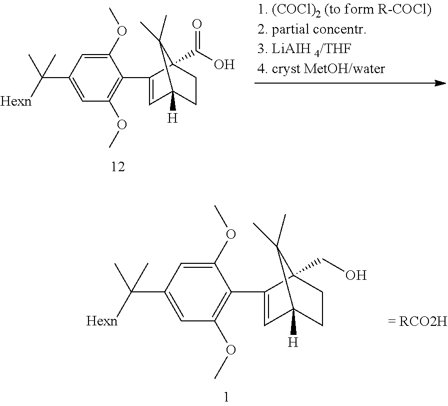

The invention further provides a crystalline structure of ((1S,4R)-2-(2,6-dimethoxy-4-(2-methyloctan-2-yl)phenyl)-7,7-dimethylbicyclo[2.2.1]hept-2-en-1-yl)methanol (HU-910) having the following unit cell: a=33.098(7) A alpha=90 deg., b=7.1740(14) A beta=124.70(3) deg. and c=25.105(5) A gamma=90 deg.

In a further aspect the invention provides a crystalline structure of ((1S,4R)-2-(2,6-dimethoxy-4-(2-methyloctan-2-yl)phenyl)-7,7-dimethylbicyclo[2.2.1]hept-2-en-1-yl)methanol (HU-910) obtainable by the process of the present invention (as defined in claims 1 to 21 hereinbelow).

In another aspect the invention provides a crystalline structure of ((1S,4R)-2-(2,6-dimethoxy-4-(2-methyloctan-2-yl)phenyl)-7,7-dimethylbicyclo[2.2.1]hept-2-en-1-yl)methanol (HU-910) obtained by the process of the invention (as defined in claims 1 to 21 hereinbelow). In some embodiments thecrystalline structure of HU-910 according to claim 20, having the following unit cell: a=33.098(7) A alpha=90 deg., b=7.1740(14) A beta=124.70(3) deg. and c=25.105(5) A gamma=90 deg.

The invention also relates to a crystalline structure of ((1S,4R)-2-(2,6-dimethoxy-4-(2-methyloctan-2-yl)phenyl)-7,7-dimethylbicyclo[2.2.1]hept-2-en-1-yl)methanol (HU-910), having the x-ray diffraction pattern as described in FIG. 1.

The invention also provides a crystalline structure of ((1S,4R)-2-(2,6-dimethoxy-4-(2-methyloctan-2-yl)phenyl)-7,7-dimethylbicyclo[2.2.1]hept-2-en-1-yl)methanol (HU-910), having the x-ray diffraction pattern as described in FIG. 1, obtained by the process of the invention (as described in claims 1 to 21 hereinbelow).

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is the crystalline x-ray scattering pattern of HU-910 obtained by the process disclosed in the present invention.

It will be appreciated that for simplicity and clarity of illustration, elements shown in the FIGURES have not necessarily been drawn to scale. For example, the dimensions of some of the elements may be exaggerated relative to other elements for clarity. Further, where considered appropriate, reference numerals may be repeated among the FIGURES to indicate corresponding or analogous elements.

### GENERAL DESCRIPTION

The invention provides the following process for the preparation of HU-910:

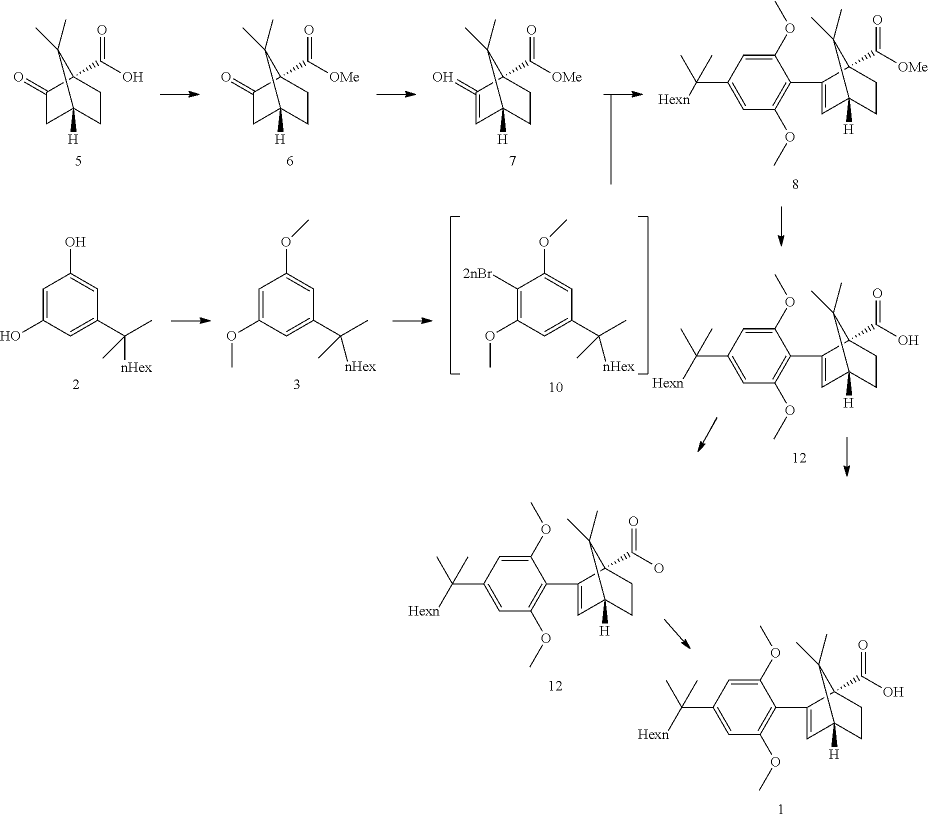

According to this aspect, the ketopinic acid esterification stage is performed with greener and safer conditions, there is no need for preparing and isolating an arylboronate reagent (the aryl zinc reagent is prepared in situ), chromatographic purification of the ester 8 is not necessary, the ester 8 is hydrolysed to the corresponding acid 12 which was purified by extractive work-up and crystallization, the acid 12 can be reduced directly but is better transformed to the corresponding acid chloride which can undergo a clean and fast reduction to give 1.

Reaction 2a: Ketopinic Methyl Ester

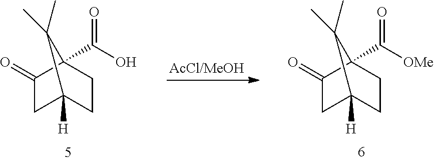

Simple Fischer esterification conditions have been developed. In situ generated HCl_{gas}/MeOH from AcCl/MeOH was selected (for related previous literature conditions: Gilbertson, Scott R.; Fu, Zice, Organic Letters, 2001, vol. 3, #2 p. 161-164: the acid is added to a solution of SOCl₂ in MeOH, which basically generates HCl. The reaction was run at RT overnight and the ester was chromatographed). Conversions >98% were achieved after ca 3-4 h at 50° C. The ester 6 was isolated after aqueous work-up. A direct solvent exchange to THF (the solvent for the next step) was not recommended as THF opening was observed. A basic extraction ensured complete removal of any remaining acid 5. The crude heptane extract was azeotroped (water and MeOH removal), exchanged to THF and introduced directly in the next step (GC a % purity >99%). More stringent conditions for example the addition of a water scavenger like trimethylorthoformate did lead to dimethyl ketal formation.

Step 2b and Triflate Coupling:

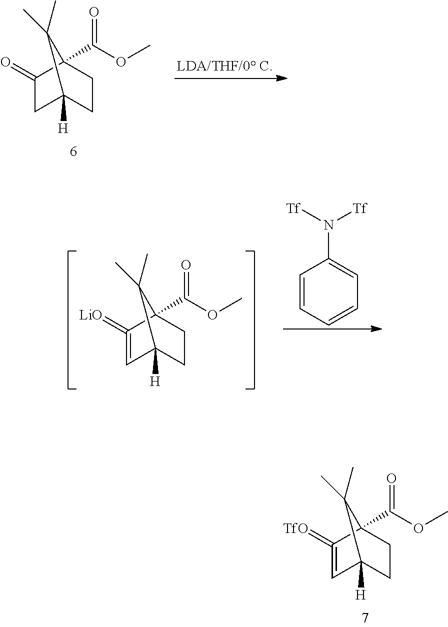

The original conditions involved the preparation of LDA at −78° C. for 1 h, deprotonation of the ester 6 at −78° C. for ca 1 h followed by addition of N-phenyltrifluoromethanesulfonimide and reaction at −78° C. for ca 1 h. The reaction sequence can be conducted at 0-5° C. ReactIR reaction monitoring indicated a feed-controlled behavior of all steps. The ester-enolate mixture and the enolate solution were found to be stable which allowed a direct dosing of LDA to the ester 6 solution. ReactIR monitoring allowed for easy IPC control. The equivalence point was determined by following the appearance of the enolate band during the deprotonation. After the equivalence point was reached, the addition of further LDA solution did dilute the ester solution and decreased its concentration which was directly identified by a decrease of the IR signal.

The crude triflate 7 solution was obtained after extractive work-up and solvent exchange to heptane. The N-phenyltrifluoromethanesulfonimide reagent by-product was removed by washing the heptane product solution with a mixture of MeOH/water. The crude heptane extract was concentrated to an oil, to give crude triflate 7 in >99a % purity by GC. The triflate 7 was introduced in the Negishi step without further purification.

Resorcinol Building Block Dimethylation

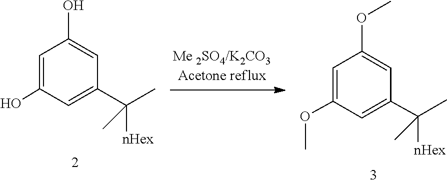

The commercially available resorcinol building block 2 (ca 97a % pure, containing alkyl side chain analogs) was dimethylated with dimethylsulfate overnight in refluxing acetone and potassium carbonate as base. After completion of the reaction, a solution of aqueous ammonia was added in order to destroy excess dimethylsulfate. Water was added and acetone was distilled off. Heptane was added and the crude product 3 was obtained after extraction, solvent switch to toluene and azeotropic drying (ca 97a %, contaminated with the chain analogs). The crude dimethyl resorcinol 3 was introduced in the next step without further purification. Distillation would be possible but does provide only a marginal purity increase.

Step 3: Negishi Coupling

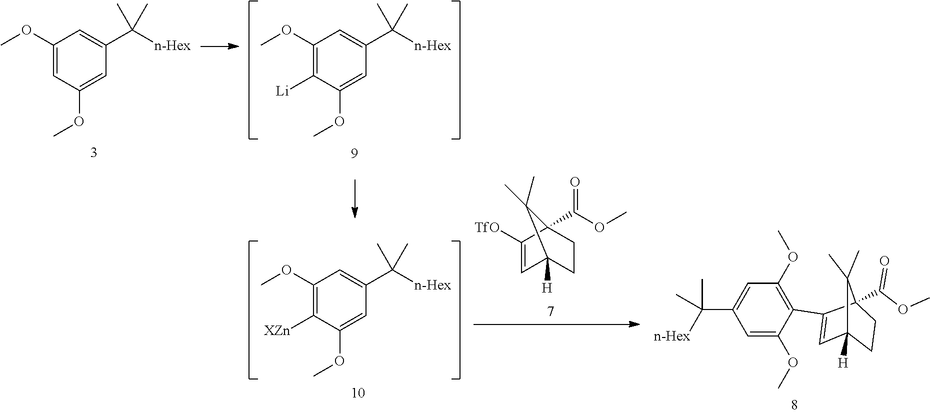

The dimethoxy resorcinol 3 deprotonation was performed at 0-15° C. The reaction is basically feed controlled. Some excess of BuLi was necessary to ensure complete deprotonation.

The THF opening by BuLi is probably partially competitive at the deprotonation temperature.

When the deprotonation was conducted at 0° C., 1.25 equiv. of BuLi/1.1 equiv. resorcinol building block 3 did provide good conversions. When the temperature was raised to 15° C., 1.3 equiv. BuLi was at the limit.

The transmetallation to the aryl zinc 10 was also feed controlled and showed a sharp drop of heat release during dosing, indicating an equivalence point where all present lithium species have been transmetallated to zinc (di-arylzinc and triarylzincate, potential butyl zinc analogs from excess BuLi). The second part of the addition led to a smaller heat release as additional zinc bromide only entered the Schlenck equilibrium.

The Negishi coupling was performed (after triflate and catalyst addition) at 0° C. for about 30 min followed by a heating ramp to RT to complete the reaction. However during the heating ramp, an inflexion point in the Tj curve was seen at ca 15° C., indicating a significant reaction heat release. The coupling was then performed at 15° C. isothermal and a 2 heat waves profile was confirmed (30 min triflate addition time). The conversion curve did show a slight inflexion point but not dramatic.

This heat release profile was shown to be depending on the amount of zinc bromide added. Lower amounts (0.6 equiv. vs 1.1 equiv.) did slow down the reaction and delayed the second wave. Excess zinc bromide was, however, detrimental to the reaction as it did lead to more side products.

Step 4a: Hydrolysis

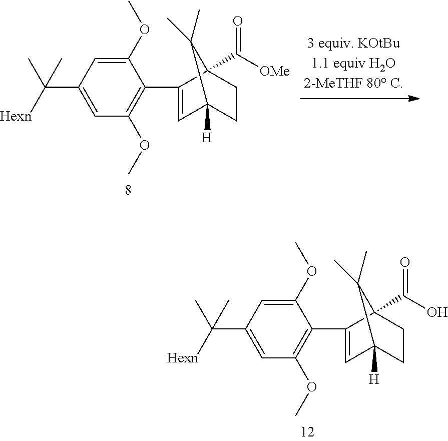

Refluxing a mixture of the ester 8, 3 equiv. KOtBu and 1.1 equiv. water in either THF or 2-MeTHF did provided the acid 12 in good yields. MeTHF was selected as it did allow for a direct extractive work-up. Most of the salts were removed during the first basic wash as the product was soluble in the organic phase due to its high lipophilicity. The solvent was then exchanged to heptane. Water and methanol were added. In this mixture, the carboxylic acid salt was soluble in the aqueous phase and all the apolar non carboxylic impurities could be removed. The aqueous product solution was filtered through an active charcoal plate. The solution was heated to 60-65° C. and was acidified by addition of HCl. The solution was cooled to 50° C. and seeded upon which crystallization did start. The suspension was cooled to RT and the acid 12 was isolated by filtration. This step provided an excellent purification and delivered the acid 12 in ca 98a % purity and 58% yield from the resorcinol building block 3. Pd content was below the LOD. Acid purification via salt formation (sodium, dicyclohexyl amine) was attempted but was not as efficient as the preferred acid isolation.

Step 4b: API—Acid Reduction

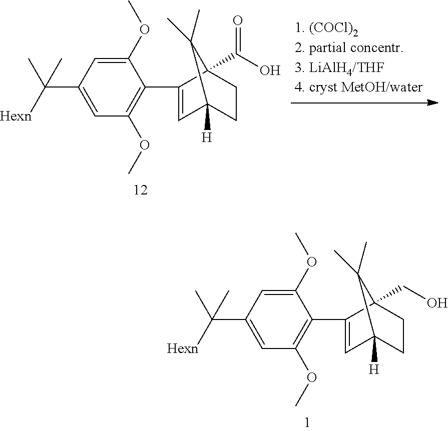

Direct reduction of the acid 12 with LAH in refluxing THF did provide the expected compound 1 in good yield. The acid reduction was replaced with a corresponding acid chloride reduction which proceeded at lower temperature and with smoother kinetics due to the higher reactivity of the substrate.

The acid chloride was prepared in situ by reaction with oxalyl chloride at RT in toluene. After completion of the reaction, CO₂, CO and HCl were removed by a partial distillation of the solution under vacuum. The reduction with LAH did proceed in a feed controlled manner with very low accumulation.

Compound 1 can be isolated by crystallization from cold heptane, ethanol/water mixtures or methano/water mixtures, preferably from ethanol/water or methanol/water, more preferably from methano/water.

Resorcinol Building Block Synthesis Through the Dimethoxybenzonitrile Route

An alternative production of the resorcinol building block 3 was developed starting from dimethoxybenzonitrile which delivered the product in >99a % GC purity and excellent overall yield.

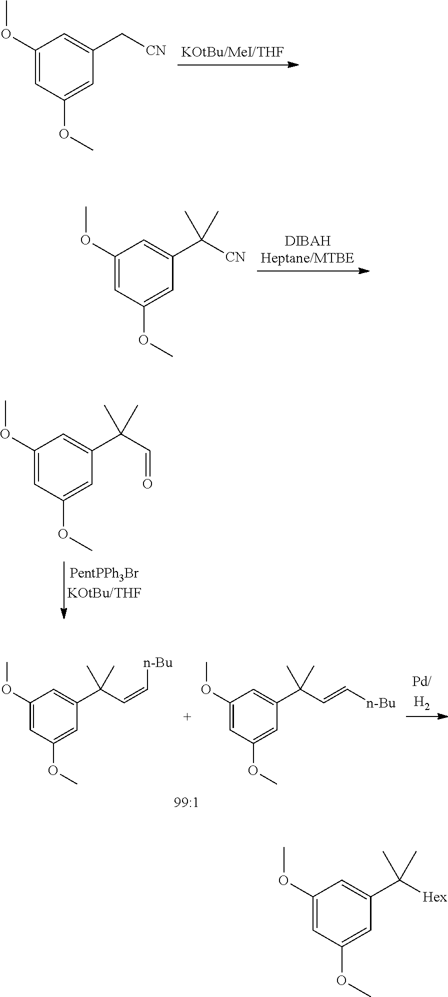

Reaction 1a: Dimethylation

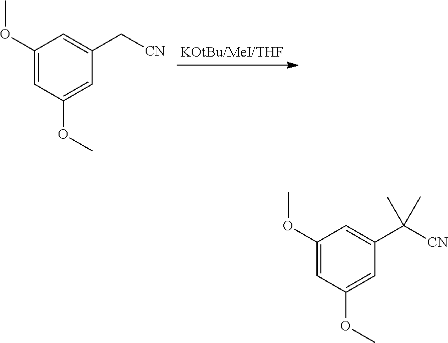

The dimethylation reaction was performed by adding a KOtBu solution in THF to a mixture of dimethoxybenzonitrile and MeI in THF at 0° C. The reaction was basically feed controlled (IPC by GC). The product was isolated after aqueous work-up and introduced directly in the reduction step. This is a clear improvement over the process described in WO2014062965 where the reaction is performed with NaH in DMF which is a known unsafe mixture. In this description, the product was purified by chromatography.

Reaction 1b: Reduction

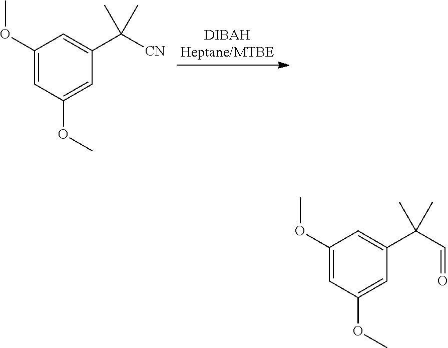

The reduction was performed at 10-20° C. in MTBE by addition of DIBAH in heptane.

Reaction 1c: Wittig

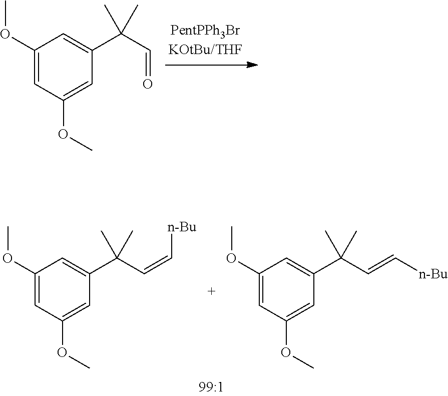

A simple Wittig reaction on the aldehyde intermediate with commercially available phosphonium salt delivered the expected product in a ca 99:1 isomeric purity. A MeOH/water/heptane work-up did provide an excellent purification method, removing TPPO and side products carried over from the previous step.

Reaction 1d: Hydrogenation

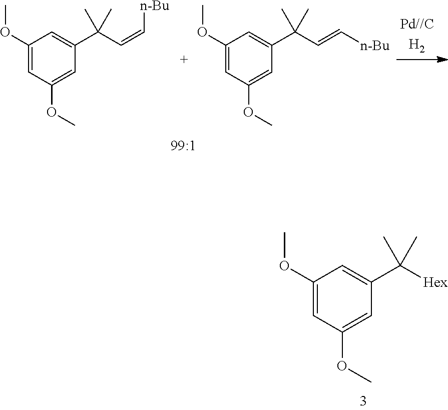

The double bond was hydrogenated under standard conditions delivering the resorcinol building block in high yield and purity (>99% purity, side chain analogs <0.1% each).

### DETAILED DESCRIPTION OF EMBODIMENTS

### (1S,4R)-7,7-dimethyl-2-oxobicyclo[2.2.1]heptane-1-carboxylic acid methyl-ester

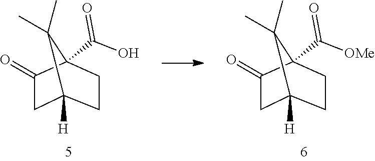

(1S,4R)-7,7-dimethyl-2-oxobicyclo[2.2.1]heptane-1-carboxylic acid (100 g, 549 mmol, Eq: 1.00) was charged in a 2 L reactor and was dissolved in Methanol (474 g, 600 mL, Eq: −). The solution was heated to 50° C. and acetyl chloride (88.0 g, 79.7 mL, 1.12 mol, Eq: 2.04) was added over 10-30 min. The reaction mixture was stirred for ca 3-4 h at 50° C. (IPC: <2% starting material). The solution was cooled to RT and added (under stirring) to a solution of NaHCO₃ (110 g, 1.31 mol, Eq: 2.39) in water (1.3 L). The biphasic mixture was concentrated under reduced pressure (170 mbar, ca 400 mL distilled). Heptane (1 L) was added. The organic phase was separated and washed with water. The aqueous phases were extracted sequentially with heptane (1 L). The organic phases were combined and concentrated under reduced pressure to give 100 g of the title compound as a crude oil (<0.1% water, 0.1% heptane, e.r. 99:1).

### Methyl (1S,4R)-7,7-dimethyl-2-(trifluoromethylsulfonyloxy)bicyclo[2.2.1]hept-2-ene-1-carboxylate

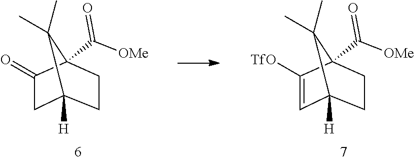 [SMILES: COC(=O)[C@]12CC[C@H](CC1=O)C2(C)C ; COC(=O)[C@]12CC[C@H](C=C1OS(=O)(=O)C(F)(F)F)C2(C)C]

A solution of methyl (1S,4R)-7,7-dimethyl-2-oxobicyclo[2.2.1]heptane-1-carboxylate (100 g, 510 mmol, Eq: 1.00) in THF (750 mL) was cooled to 0° C. LiHMDS 1.0 M in THF (530 g, 595 mL, 633 mmol, Eq: 1.24) was added dropwise over 30 min. The addition funnel was washed with THF (53 mL). After 30 min reaction (the deprotonation can be followed by inline FTIR with a ReactIR system), a solution of 1,1,1-trifluoro-N-phenyl-N-(trifluoromethyl-sulfonyl)methanesulfonamide (191 g, 535 mmol, Eq: 1.05) in THF (500 mL) was added dropwise over 30 min (the triflate formation can be followed by inline FTIR with a ReactIR system). The addition funnel was washed with THF (53 mL). After 30 min at 0-5° C. (IPC by GC: <1% ketoester starting material), the reaction mixture was added to a solution of NH₄Cl (376 g, 7.03 mol, Eq: 13.8) in water (1.12 L). The biphasic mixture was warmed to RT. The aqueous phase was separated and extracted with MTBE (638 mL). The organic phases were combined and concentrated under reduced pressure to give 300 g of a crude oil. The oil was taken up in a mixture of MeOH (511 mL), water (128 mL) and heptane (1.3 L). The aqueous phase was separated and re-extracted twice with heptane (1.3 L). The organic phases were washed sequentially 4 times with 0.64 L of a 4:1 v/v MeOH/water mixture. The organic phases were combined and concentrated under reduced pressure to give 145 g of the title compound (99.5a % GC, MeOH<0.1%, heptane<0.1%, water<0.1%).

### 1,3-dimethoxy-5-(2-methyloctan-2-yl)benzene

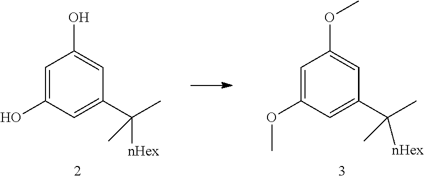

Potassium carbonate (233 g, 1.68 mol, Eq: 3.98) and commercially available 5-(2-methyloctan-2-yl)benzene-1,3-diol (100 g, 423 mmol, Eq: 1.00) charged in the reactor, followed by acetone (1.27 L). The suspension was heated to 50° C. A solution of dimethylsulfate (144 g, 109 mL, 1.14 mol, Eq: 2.7) in acetone (211 mL) was added over 1 h. After 16 h reaction (IPC: starting material: nd; monomethyl intermediate: 0.09%; product 97.3%), 25% ammonium hydroxide (41.0 g, 45 mL, 601 mmol, Eq: 1.42) was added. After 30 min at 50° C. (IPC: dimethyl sulfate<LOD), water (1.27 L) was added and the reaction mixture was concentrated under reduced pressure (100-300 mbar, 1.2-1.5 L distilled) and cooled to RT. Heptane (1.27 L) was added. The organic phase was separated and washed with water (850 mL). The organic phase was concentrated under reduced pressure to an oil. The crude oil was azeotroped with toluene (200 mL) to give 114 g of the title compound as a crude oil (contains 6% w/w residual toluene).

### Methyl (1S,4R)-2-(2,6-dimethoxy-4-(2-methyloctan-2-yl)phenyl)-7,7-dimethylbicyclo[2.2.1]hept-2-ene-1-carboxylate

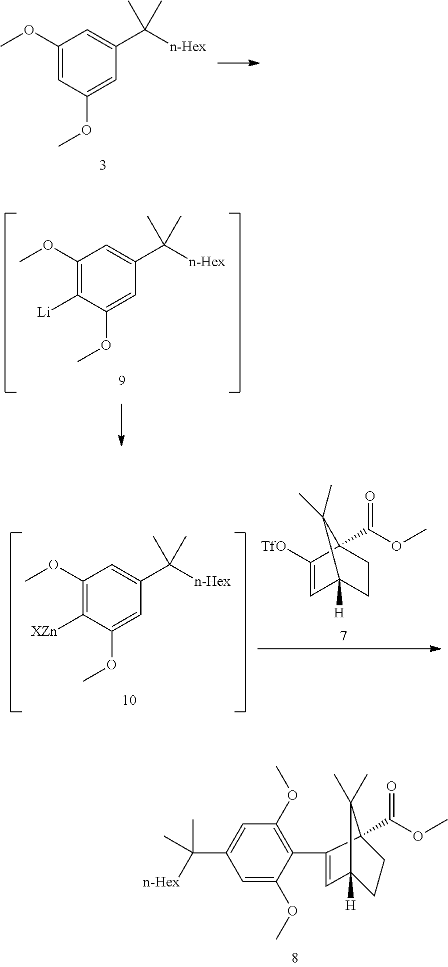

1,3-dimethoxy-5-(2-methyloctan-2-yl)benzene (2.25 kg, 8.3 mol, Eq: 1.1) was dissolved in THF (21 L). The solution was cooled to 15° C. 1.6 M BuLi in hexane (4.1 kg, 10.2 mol, Eq: 1.36) was added over 35 min. The addition funnel was washed with THF (1 L). After 1.5 h at 15° C., a solution of 30% zinc bromide in THF (7.5 kg, 6.25 L, 9.99 mol, Eq: 1.33) was added over 35 min. The addition funnel was washed with THF (1 L). After 45 min, a tetrakis(triphenyl-phosphine)palladium (431 g, 373 mmol, Eq: 0.0495) was added in one portion. After 15 min, a solution of (1S,4R)-methyl 7,7-dimethyl-2-(trifluoromethylsulfonyloxy)bicyclo[2.2.1]hept-2-ene-1-carboxylate (2.5 kg, 7.54 mol, Eq: 1.00) in THF (9.25 L) was added over 1 h. The reaction mixture was stirred 50 min at 15° C., then overnight at 25° C. The reaction mixture was cooled to 15° C. and a mixture of 25% HCl_{aq} (2.4 kg, 2 L, 16.5 mol, Eq: 2.18) and water (30 L) was added. Heptane (30 L) was added. The organic phase was separated and washed twice with water (2×40 L, pH last water phase: 5-6). The organic phase was concentrated under reduced pressure and dried azeotropically with heptane (total 80 L). The resulting crude solution was polish filtered and concentrated to dryness to give 3.73 kg of a viscous oil. The crude product was purified by chromatography (38 kg silica gel, 50 L fractions, 9:1 heptane/toluene up to fraction 10, 4:1 heptane/toluene up to fraction 26 and 1:1 heptane/toluene up to fraction 41). The fractions 24-41 were combined and concentrated to dryness to give 2.68 kg of the title compound (6% residual toluene, 0.04% residual heptane).

### ((1S,4R)-2-(2,6-dimethoxy-4-(2-methyloctan-2-yl)phenyl)-7,7-dimethylbicyclo[2.2.1]hept-2-en-1-yl)methanol Via Ester Reduction

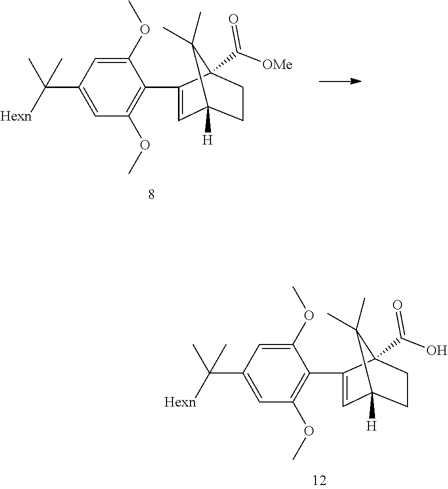

Chromatographed (1S,4R)-methyl 2-(2,6-dimethoxy-4-(2-methyloctan-2-yl)phenyl)-7,7-dimethylbicyclo[2.2.1]hept-2-ene-1-carboxylate (2.5 kg, 5.65 mol, Eq: 1.00) was dissolved in THF (25 L). The solution was heated to 40° C. 1 M LiAlH₄ in THF (6 L, 6.00 mol, Eq: 1.06) was added over 45 min. After 6 h at 40° C. (IPC control), acetone (1.58 kg, 2 L, 27.2 mol, Eq: 4.82) was added over 45 min (exotherm!). After 20 min, the reaction mixture was cooled to RT. A mixture of 25% HCl_{aq} (3.71 kg, 25.4 mol, Eq: 4.5) and water (20 L) was added over 45 min. After 45 min, water (10 L) and heptane (20 L) were added. The organic phase was separated and washed with a mixture of 25% HCl_{aq} (1.2 kg, 1 L, 8.23 mol, Eq: 1.46) and water (20 L), then twice with water (2×20 L, pH last water phase: 5). The organic phase was concentrated to dryness under reduced pressure to give 1.98 kg of crude product. The crude oil was dissolved in ethanol (10 L) and concentrated again (THF, heptane and toluene<0.1%). The crude oil was dissolved in ethanol (10 L) and polish filtered. The filtrate was cooled to 0-5° C. and water (2.5 L) was added over 10 min to give a milky emulsion. The emulsion was seeded. The crystallization started and water (2.5 L) was added over 10 min. The suspension was stirred overnight and was filtered. The filter cake was washed with a 1:1 ethanol/water mixture (3 L) and was dried at 40° C./3 mbar overnight to give 1.65 kg of the title compound as white crystals.

### Negishi-Hydrolysis Sequence

### Example 1

### a) Methyl (1S,4R)-2-(2,6-dimethoxy-4-(2-methyloctan-2-yl)phenyl)-7,7-dimethylbicyclo[2.2.1]hept-2-ene-1-carboxylate

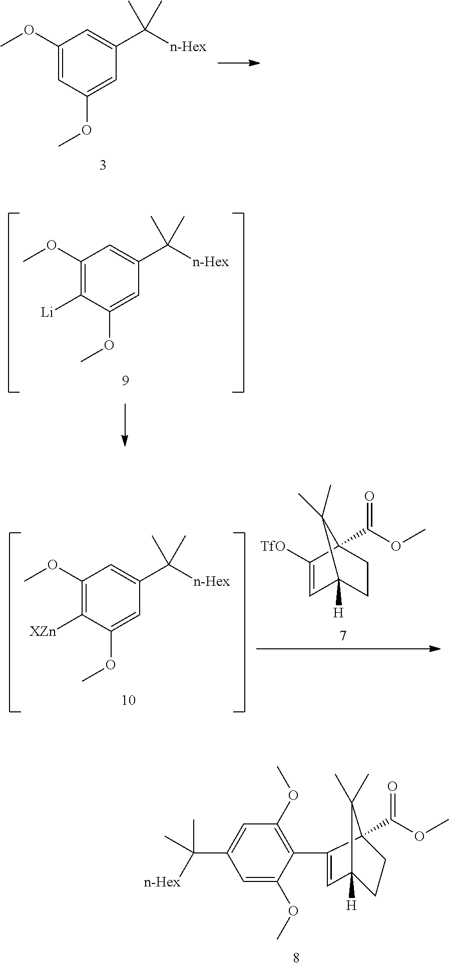

1,3-dimethoxy-5-(2-methyloctan-2-yl)benzene (59.1 g, 197 mmol, Eq: 1.1) was dissolved in THF (444 g, 500 mL, Eq: −). The solution was cooled to 10-15° C. A 15.2% BuLi solution in hexane (104 g, 247 mmol, Eq: 1.38) was added dropwise over 15 min. The addition funnel was washed with heptane (20 mL). After 1 h reaction at 15° C., a 30% zinc bromide solution in THF (130 g, 108 mL, 171 mmol, Eq: 0.957) was added dropwise over 15 min. The addition funnel was washed with THF (17.8 g, 20.0 mL, Eq: −). After 50 min at 15° C., a suspension of tetrakis(triphenylphosphine)palladium (8.9 g, 7.7 mmol, Eq: 0.0430) in THF (20 mL) was added in one portion. The feed vessel was washed with THF (10 mL). After 15 min at 15° C., a solution of methyl (1 S,4R)-7,7-dimethyl-2-(trifluoromethylsulfonyloxy)bicyclo[2.2.1]hept-2-ene-1-carboxylate (60 g, 179 mmol, Eq: 1.00) in THF (180 mL) was added dropwise over 20 min. The addition funnel was washed with THF (20 mL). After 20 min at 15° C., the reaction mixture was heated to 25° C. and stirred overnight at 25° C. (IPC 50 min RT: ca 1% triflate left). The reaction mixture was cooled to 5° C. and a mixture of 25% hydrochloric acid (55 g, 45.8 mL, 377 mmol, Eq: 2.11) in water (425 mL) was added dropwise. Heptane (530 mL) was added. The organic phase was separated and washed twice with water (500 mL). The organic phase was solvent exchanged to heptane (750 mL heptane in total, final volume 250 mL). The resulting suspension was first filtered on a glass sintered filter, then filtered through an active charcoal filter and finally concentrated under reduced pressure to give in total 87.9 g of crude title compound as a viscous oil. This crude product can be purified by chromatography (silica gel, 9:1 heptane/toluene to 1:1 heptane/toluene) but is best introduced directly in the following hydrolysis step where the corresponding acid (or one of its salts) can be easily purified.

### b) (1S,4R)-2-(2,6-dimethoxy-4-(2-methyloctan-2-yl)phenyl)-7,7-dimethylbicyclo[2.2.1]hept-2-ene-1-carboxylic Acid

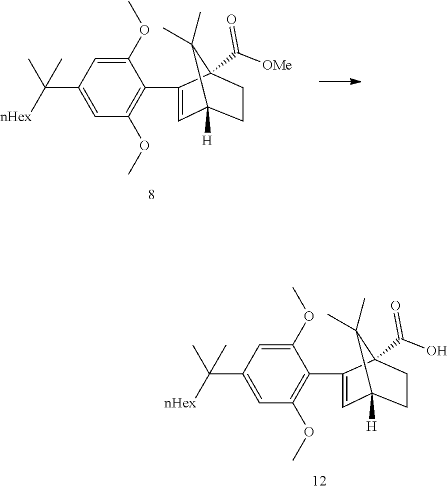

Potassium tert-butoxide (11.4 g, 102 mmol, Eq: 3.0) was suspended in 2-MeTHF (27 mL). A solution consisting of (1S,4R)-methyl 2-(2,6-dimethoxy-4-(2-methyloctan-2-yl)phenyl)-7,7-dimethylbicyclo[2.2.1]hept-2-ene-1-carboxylate (15 g crude ester from previous step, taken as 100%, 33.9 mmol, Eq: 1.00), 2-MeTHF (103 mL) and water (672 mg, 672 μL, 37.3 mmol, Eq: 1.1) was added over 15 min. The reaction mixture was heated to reflux overnight. Heptane (79 mL) was added. The mixture was solvent exchanged to heptane (200-90 mbar/50° C.) at constant volume. The resulting suspension was treated with a mixture consisting of MeOH (79 mL) and water (20 mL). The biphasic mixture was polish filtered. The aqueous phase was separated and washed with heptane (79 mL). The organic phases were extracted sequentially twice with a 4:1 MeOH/water mixture (2×40 mL). The aqueous phases were combined and the pH was adjusted to ca 1 by addition of 25% HCl_{aq} (15.2 g, 13.5 mL, 104 mmol, Eq: 3.07). The crystallization started spontaneously. After 1 h at RT, the suspension was filtered. The filter cake was washed with a 1:1 MeOH/water mixture (40 mL), twice with water (35 mL) and dried at 50° C./1 mbar to give 8.5 g of the title compound as white crystals (>95% purity by GC).

### Example 2

### a) Methyl (1S,4R)-2-(2,6-dimethoxy-4-(2-methyloctan-2-yl)phenyl)-7,7-dimethylbicyclo[2.2.1]hept-2-ene-1-carboxylate

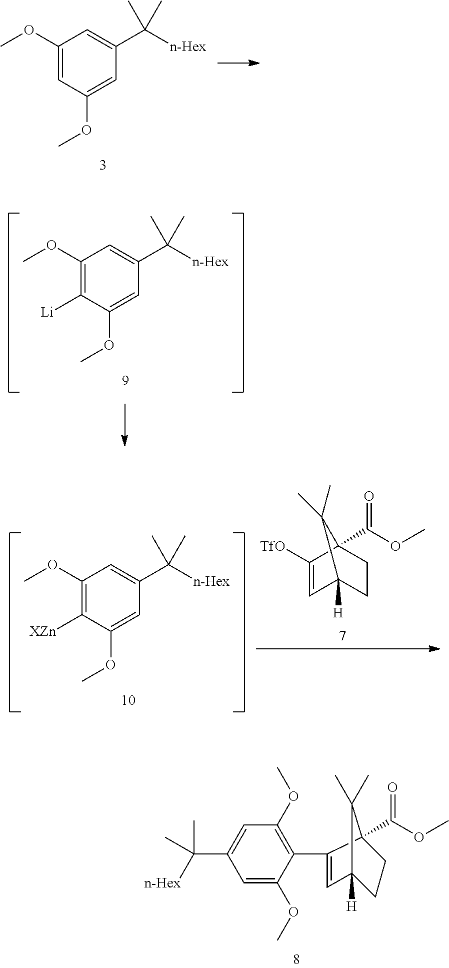

1,3-dimethoxy-5-(2-methyloctan-2-yl)benzene (54.0 g, 199 mmol, Eq: 1.1) was dissolved in THF (500 mL). The solution was cooled to ca 15° C. and 1.6 M BuLi in hexane (100 g, 250 mmol, Eq: 1.38) was added over 15 min. The addition funnel was washed with heptane (20 mL). After 1 h at 15° C., 30% zinc bromide (130 g, 108 mL, 173 mmol, Eq: 0.957) was added over 15 min. The addition funnel was washed with THF (20 mL). After 45 min at 15° C., a suspension of tetrakis(triphenylphosphine)palladium (8.8 g, 7.62 mmol, Eq: 0.0421) in THF (20 mL) was added in one portion. The feed tank was washed with THF (10 mL). A solution of (1S,4R)-methyl 7,7-dimethyl-2-(trifluoromethylsulfonyloxy)bicyclo[2.2.1]hept-2-ene-1-carboxylate (60 g, 181 mmol, Eq: 1.00) in THF (200 mL) was added over 15 min. The reaction mixture was stirred 50 min at 15° C. and 90 min at RT. The reaction mixture was cooled to 15° C. and a solution consisting of 25% hydrochloric acid (57.6 g, 48.0 mL, 395 mmol, Eq: 2.18) and water (700 mL) was added. The biphasic mixture was stirred for 30 min. Heptane (720 mL) was added. The organic phase was separated and washed twice with water (2×700 mL, pH last aqueous phase 5-6). The organic phases were combined and solvent exchanged to heptane. The resulting suspension was filtered. The filter cake was washed with heptane (200 mL). The filtrate was concentrated under reduced pressure to give 92 g of the crude title compound as an oil (ca 75a % by GC).

### b) (1S,4R)-2-(2,6-dimethoxy-4-(2-methyloctan-2-yl)phenyl)-7,7-dimethylbicyclo[2.2.1]hept-2-ene-1-carboxylic Acid

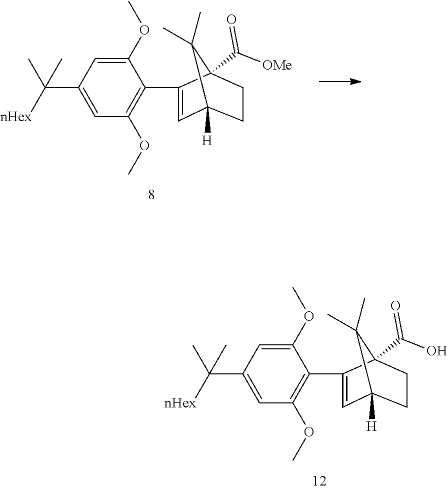

Potassium tert-butoxide (70.0 g, 624 mmol, Eq: 3.0) was suspended in 2-MeTHF (121 mL) at RT. A solution consisting of methyl (1S,4R)-2-(2,6-dimethoxy-4-(2-methyloctan-2-yl)phenyl)-7,7-dimethylbicyclo[2.2.1]hept-2-ene-1-carboxylate (92 g crude ester from previous step, taken as 100%, 208 mmol, Eq: 1.00), 2-MeTHF (680 mL) and water (4.12 g, 4.12 mL, 229 mmol, Eq: 1.1) was added and the reaction mixture was heated at reflux overnight. After reaction completion, 470 mL of solvent were distilled. Heptane (485 mL) was added. The resulting mixture was solvent exchanged to heptane at constant volume (ca 1.45 L). A mixture of MeOH (485 mL) and water (121 mL) was added. The aqueous phase was separated and extracted with heptane (485 mL). The organic phases were twice washed sequentially with a 4:1 MeOH/water mixture (2×242 mL). The aqueous phases were combined (total volume ca 1.25 L). 25% HCl (98.1 g, 87.2 mL, 673 mmol, Eq: 3.24) was added over 20 min to adjust the pH to ca 1-1.5. The product started to crystallize. The suspension was stirred 1 h at RT and was filtered. The filter cake was washed with a 1:1 MeOH/water mixture (200 mL), then twice with water (2×200 mL). The filter cake was dried under reduced pressure (50° C./5 mbar) until constant weight to give 60.5 g of the title compound as white crystals (>95% purity by GC).

### (1S,4R)-2-(2,6-dimethoxy-4-(2-methyloctan-2-yl)phenyl)-7,7-dimethylbicyclo[2.2.1]hept-2-ene-1-carboxylic Acid

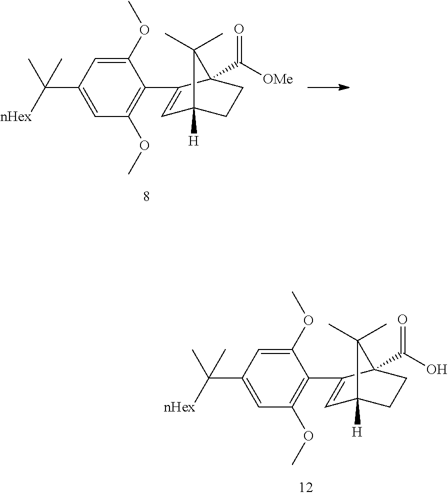

Chromatographed (1S,4R)-methyl 2-(2,6-dimethoxy-4-(2-methyloctan-2-yl)phenyl)-7,7-dimethylbicyclo[2.2.1]hept-2-ene-1-carboxylate (20 g, 45.2 mmol, Eq: 1.00) was dissolved in MeTHF (200 mL). KOtBu (15.2 g, 136 mmol, Eq: 3) was added, followed by water (896 mg, 896 μL, 49.7 mmol, Eq: 1.1). The reaction mixture was heated to reflux for >3 h (IPC). The reaction mixture was cooled to RT. 1 M HCl_{aq} (181 mL, 181 mmol, Eq: 4) and 2-MeTHF (54 mL) were added. The organic phase was separated, washed with water (100 mL), dried over Na₂SO₄ and concentrated to dryness to give 18.7 g of the title compound.

### ((1S,4R)-2-(2,6-dimethoxy-4-(2-methyloctan-2-yl)phenyl)-7,7-dimethylbicyclo[2.2.1]hept-2-en-1-yl)methanol Via Acid Reduction

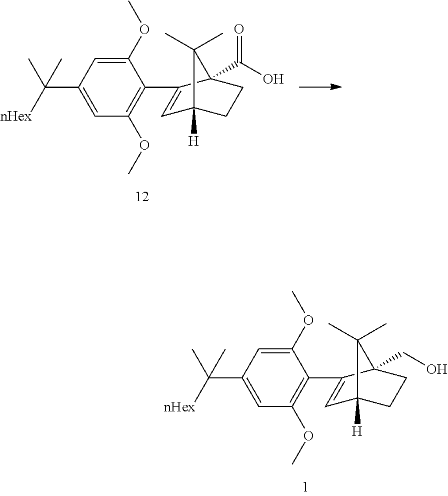

(1S,4R)-2-(2,6-dimethoxy-4-(2-methyloctan-2-yl)phenyl)-7,7-dimethylbicyclo[2.2.1]hept-2-ene-1-carboxylic acid (9.52 g, 22.2 mmol, Eq: 1.00) was dissolved in toluene (100 mL). The solution was heated to 50-55° C. and 1 M LiAlH₄ in THF (22 mL, 22.0 mmol, Eq: 0.990) was added dropwise within ca 15 min. After 3 h reaction, the reaction mixture was cooled to RT and added to a mixture of 25% aqueous HCl (19.4 g, 133 mmol, Eq: 6) and water (100 mL). After 40 min stirring, heptane (120 mL) was added. The organic phase was separated and washed twice with water (2×100 mL). The organic phases were combined and concentrated under reduced pressure to give 9.7 g of an oil which was re-dissolved in MeOH (100 mL) and concentrated at atmospheric pressure to give 9.14 g of the crude title compound as a colorless oil. The crude product was dissolved in MeOH (35 mL). Water (4 mL) was added and the mixture was heated to 50° C. to give a milky turbid mixture. This was cooled to 0° C. within 1 h (seeding performed at 40° C.) during which the product started to crystallize. Water (4 mL) was added, the suspension was stirred for 3 h at 0° C. and was filtered. The filter cake was washed with a 1:1 MeOH/water mixture (10 mL) and was dried under reduced pressure (40-45° C./2 mbar) until constant weight to give 8.28 g of the title compound as a white crystalline powder.

### ((1S,4R)-2-(2,6-dimethoxy-4-(2-methyloctan-2-yl)phenyl)-7,7-dimethylbicyclo[2.2.1]hept-2-en-1-yl)methanol Via Acid Chloride Reduction

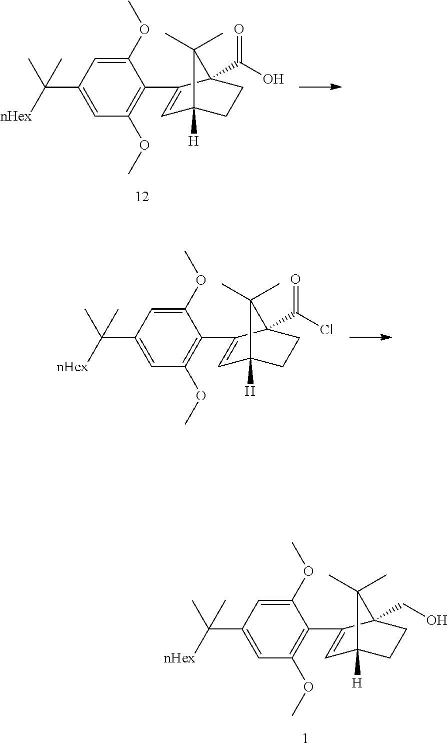

(1S,4R)-2-(2,6-dimethoxy-4-(2-methyloctan-2-yl)phenyl)-7,7-dimethylbicyclo[2.2.1]hept-2-ene-1-carboxylic acid (50 g, 117 mmol, Eq: 1.00) was dissolved in toluene (500 mL). DMF (142 mg, 150 μL, 1.94 mmol, Eq: 0.0166) was added. Oxalyl chloride (16.4 g, 11.3 mL, 127 mmol, Eq: 1.09) was added dropwise at 25° C. The addition funnel was washed with toluene (10 mL). The reaction mixture was stirred overnight at RT (the reaction is usually finished in <2 h). About 100 mL of solvent was distilled of under reduced pressure to remove HCl and CO₂. A 1 M LiAlH₄ solution in THF (111 g, 117 mmol, Eq: 1.00) was added over 30 min and the addition funnel was washed with toluene (10 mL). After completion of the reaction (IPC by GC, <2 h usually), the reaction mixture was added over 45 min to a mixture of 25% HCl (110 g, 754 mmol, Eq: 6.47) and water (400 mL) at 10° C. Heptane (600 mL) was added (the resulting mixture could be kept overnight without noticable degradation). The organic phase was separated and washed twice with water (2×500 mL). The organic phase was concentrated under reduced pressure (50° C./150-70 mbar). The crude solution was solvent exchanged to methanol (resulting volume 1 L) and cooled to 0° C. Water was added (100 mL) and the resulting clear solution was seeded. The product started to crystallize. The suspension was stirred overnight at 0° C. Water (200 mL) was added over 2 h. The suspension was stirred for 4 h at 0° C. and filtered. The filter cake was washed with a 1:1 MeOH/water mixture (100 mL) and dried at 45° C./1 mbar to give 44 g of the title compound as white crystals.

High Purity API from High Purity Resorcinol Building Block

### 2-(3,5-dimethoxyphenyl)-2-methylpropanenitrile

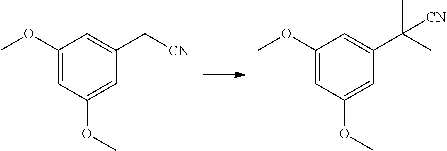

A solution of KOtBu (48.5 g, 423 mmol, Eq: 3.0) in THF (300 mL) was cooled to −15° C. A solution of 2-(3,5-dimethoxyphenyl)acetonitrile (25 g, 141 mmol, Eq: 1.00) and iodomethane (80.9 g, 35.5 mL, 564 mmol, Eq: 4.0) in THF (85 mL) was added dropwise over 55 min at −15° C. to −10° C. THF (100 mL) was added (IPC by GC: 90% Prod, 3.5% monomethylated intermediate, 5.2% starting material). The suspension was warmed over 20 min to 20° C. and stirred for 1h45 (IPC: no significant change). THF (100 mL) was added, followed by KOtBu (8.08 g, 70.5 mmol, Eq: 0.5) and iodomethane (20.2 g, 8.91 mL, 141 mmol, Eq: 1.0). After 1 h at 20° C. (IPC GC: 99% Prod, monomethyl intermediate and starting material n.d.), the reaction mixture was added to a mixture of MTBE (167 mL) and water (167 mL). The organic phase was separated, washed twice with 25% aqueous NaCl (167 mL), dried over Na₂SO₄, filtered and concentrated under reduced pressure (45° C./10mabr) to give 27.3 g of the title compound as an oil.

### 2-(3,5-dimethoxyphenyl)-2-methylpropanenitrile

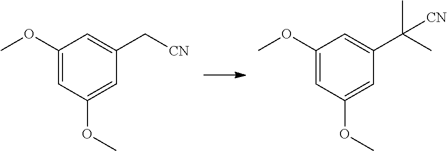

2-(3,5-dimethoxyphenyl)acetonitrile (100 g, 564 mmol, Eq: 1.00) was dissolved in THF (500 mL). The solution was cooled to 0° C. and iodomethane (324 g, 142 mL, 2.26 mol, Eq: 4.0) was added. 1 M Potassium tert-butoxide solution in THF (1.69 L, 1.69 mol, Eq: 3) was added dropwise at 0° C.−2° C. over 60 min (conversion>99% by GC after end addition). After 1 h at 0° C., the reaction mixture was added to a stirred mixture of MTBE (1.5 L) and water (750 mL). The organic phase was separated, washed twice with 25% aqueous NaCl (750 mL), dried over Na₂SO₄, filtered and concentrated under reduced pressure to give 115.8 g of the title compound as an oil (98.5a % by GC).

### 2-(3,5-dimethoxyphenyl)-2-methylpropanal

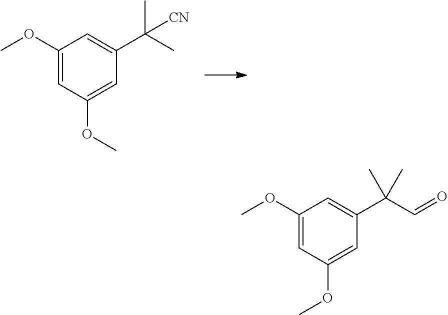

2-(3,5-dimethoxyphenyl)-2-methylpropanenitrile (10 g, 46.9 mmol, Eq: 1.00) was dissolved in MTBE (50 mL). The clear solution was cooled to 10° C. and 1 M DIBAL-H in heptane (56.3 mL, 56.3 mmol, Eq: 1.2) was added dropwise over 30 min. The reaction mixture was warmed to 20° C. over 30 min. After 2 h at 20° C., the reaction mixture was added to a cold (0° C.) mixture of heptane (50 mL) and 2 M aqueous HCl (100 mL). The water phase was separated and extracted with heptane (50 mL). The organic phases were washed twice sequentially with 2 M aqueous HCl (2×50 mL), then half saturated aqueous NaHCO₃ (50 mL) and half saturated aqueous NaCl (50 mL). The organic phases were combined, dried over Na₂SO₄, filtered and concentrated under reduced pressure to give 10.3 g of the crude title compound as an oil (97a % GC).

### (Z/E)-1,3-dimethoxy-5-(2-methyloct-3-en-2-yl)benzene

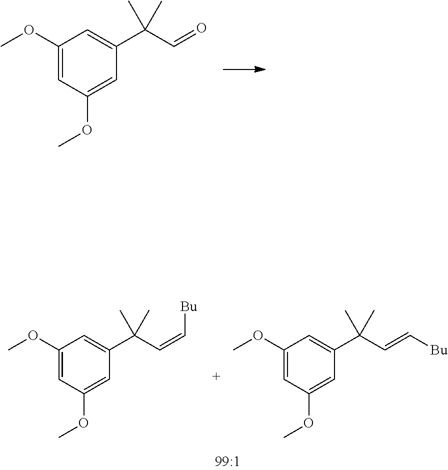

Pentyltriphenylphosphonium bromide (20.5 g, 48.7 mmol, Eq: 1.1) and 2-(3,5-dimethoxyphenyl)-2-methylpropanal (9.5 g, 44.2 mmol, Eq: 1.00) were charged in the reactor, followed by THF (48 mL). The suspension was cooled to 0° C. and a solution of KOtBu (5.57 g, 48.7 mmol, Eq: 1.1) in THF (48 mL) was added dropwise over 30 min at 0°−5° C. The reaction mixture was stirred for 50 min (98% conversion by GC). Additional KOtBu (248 mg, 2.21 mmol, Eq: 0.05) was added in one portion (color change from a beige to a yellow suspension). After 1h45 at 0° C. (GC aldehyde n.d.), the reaction mixture was poured on a stirred mixture of water (48 mL), heptane (95 mL) and 70:30 methanol/water mixture (95 mL). The aqueous phase was separated and extracted with heptane (48 mL). The organic phases were washed 3 times sequentially with a 70:30 methanol/water mixture (95 mL). The organic phases were combined, dried over MgSO₄ and concentrated under reduced pressure to give 11.1 g of the crude title compound as an oil (97.5a % Z product:1.2% E isomer).

### 1,3-dimethoxy-5-(2-methyloctan-2-yl)benzene

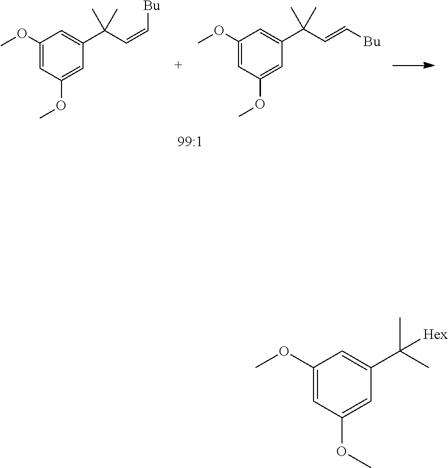

(Z)-1,3-dimethoxy-5-(2-methyloct-3-en-2-yl)benzene (9.5 g, 35.3 mmol, Eq: 1.00) was charged in the reactor, followed by AcOEt (135 g, 150 mL, Eq: −) and 10% Pd/C E101 N/D EvoniK (1.9 g, 1.79 mmol, Eq: 0.0506). The hydrogenation was performed at 30° C. and 4 bar H₂. After 18 h the reaction mixture was filtered and the filtrate was evaporated at 45° C. and concentrated under reduced pressure to give 9.1 g of the title compound as an oil (98.8a % GC).

Negishi-Hydrolysis Sequence

### a) Methyl (1S,4R)-2-(2,6-dimethoxy-4-(2-methyloctan-2-yl)phenyl)-7,7-dimethylbicyclo[2.2.1]hept-2-ene-1-carboxylate

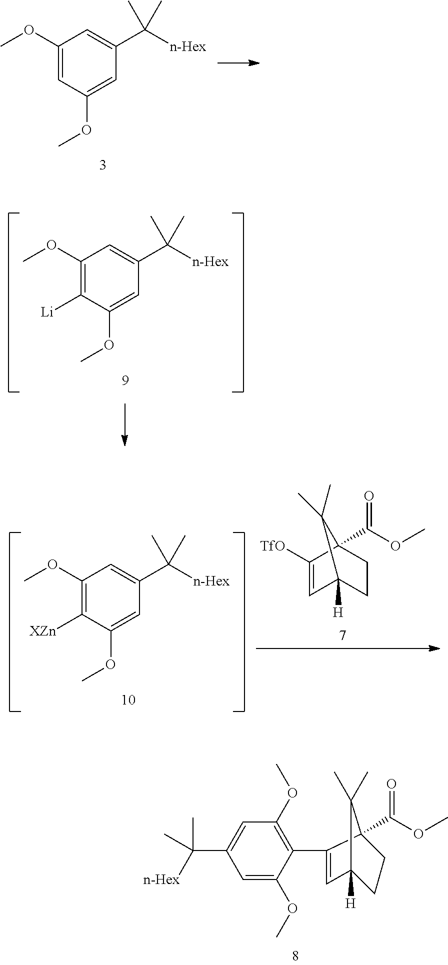

1,3-dimethoxy-5-(2-methyloctan-2-yl)benzene (7.99 g, 29.9 mmol, Eq: 1.10) was dissolved in THF (75 mL). The solution was cooled to 10-15° C. and 1.6 M BuLi in hexane (23.4 mL, 37.5 mmol, Eq: 1.38) was added over 15 min keeping the temperature between 10-15° C. After 1 h at 15° C., 30% zinc bromide in THF (19.7 g, 16.4 mL, 26.0 mmol, Eq: 0.957) was added over 15 min. After 50 min at 15° C., tetrakis(triphenylphosphine)palladium (1.36 g, 1.17 mmol, Eq: 0.043) was added in one portion. After 10 min, a solution of (1S,4R)-methyl 7,7-dimethyl-2-(trifluoromethylsulfonyloxy)bicyclo[2.2.1]hept-2-ene-1-carboxylate (9.0 g, 27.1 mmol, Eq: 1.00) in THF (30 mL) was added over 20 min. The reaction mixture was stirred 15 min at 15° C. and 1 h at 25° C. The reaction was quenched with a solution of 25% aqueous HCl (8.31 g, 6.93 mL, 57.0 mmol, Eq: 2.1) in water (60.0 g, 60 mL, Eq: −). Heptane (80 mL) was added. The crude biphasic mixture was polish filtered. The aqueous phase was separated and extracted with heptane (20 mL). The organic phases were washed twice sequentially with water (2×60 mL). The combined organic phases were concentrated under reduced pressure. The crude oil was taken up in n-heptane (40 mL), polish filtered and concentrated under reduced pressure to give 14 g of the crude title compound as an oil (67a % by GC) which was introduced in the next step without purification.

### b) (1S,4R)-2-(2,6-dimethoxy-4-(2-methyloctan-2-yl)phenyl)-7,7-dimethylbicyclo[2.2.1]hept-2-ene-1-carboxylic Acid

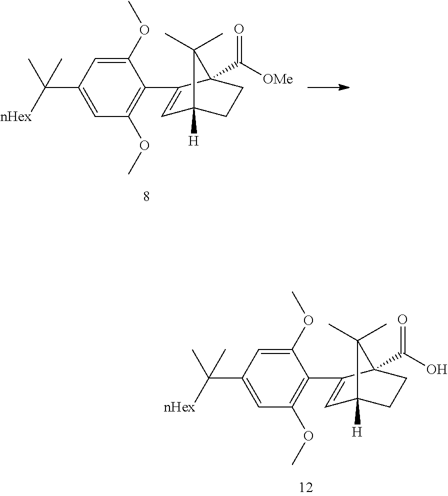

Potassium tert-butoxide (10.3 g, 91.5 mmol, Eq: 3.0) was suspended in 2-MeTHF (24.5 mL). A solution consisting of methyl (1S,4R)-2-(2,6-dimethoxy-4-(2-methyloctan-2-yl)phenyl)-7,7-dimethylbicyclo[2.2.1]hept-2-ene-1-carboxylate (13.5 g from previous step, taken as 100%, 30.5 mmol, Eq: 1.00), 2-MeTHF (86 mL) and water (605 mg, 605 μL, 33.5 mmol, Eq: 1.1) was added over 15 min at RT. The reaction mixture was heated to reflux. After completion of the reaction (3 h), the reaction mixture was cooled to 10° C. and added to water (68 mL). Heptane (270 mL) was added and the reaction mixture was concentrated (50° C./200-90 mbar) to ca 100 mL to remove 2-MeTHF. MeOH (68 mL) and water (17 mL) were added. The aquous phase was separated and extracted with heptane (270 mL). The organic phases were washed sequentially twice with a 4:1 MeOH/water (2×42 mL). The combined aqueous phases were polish filtered and heated to 50° C. The pH was adjusted to 1-2 with 25% HCl (14.2 g, 11.9 mL, 97.6 mmol, Eq: 3.20). The turbid solution was seeded. The suspension was cooled to RT over 1 h and stirred for 2 h. The suspension was filtered. The filter cake was washed with a 1:1 MeOH/water mixture (20 mL) and dried under reduced pressure (50° C./5 mbar) until constant weight to give 8.1 g of the title compound as white crystals.

### ((1S,4R)-2-(2,6-dimethoxy-4-(2-methyloctan-2-yl)phenyl)-7,7-dimethylbicyclo[2.2.1]hept-2-en-1-yl)methanol

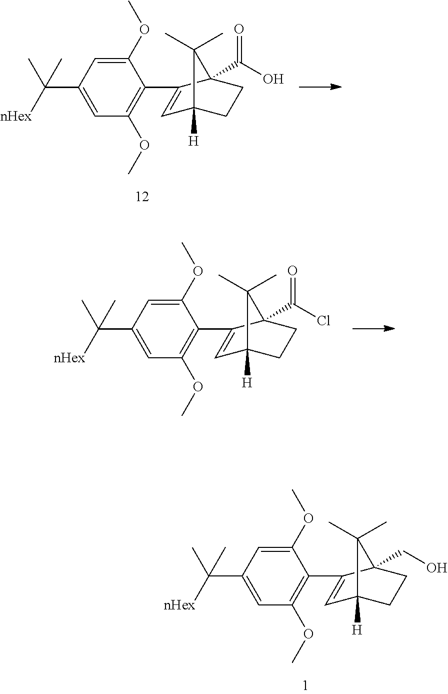

(1 S,4R)-2-(2,6-dimethoxy-4-(2-methyloctan-2-yl)phenyl)-7,7-dimethylbicyclo[2.2.1]hept-2-ene-1-carboxylic acid (7.5 g, 17.0 mmol, Eq: 1.00) was dissolved in toluene (75 mL) at 20° C. DMF (24.9 mg, 26.2 μL, 339 μmol, Eq: 0.02) was added in one portion. Oxalyl dichloride (2.42 g, 1.64 mL, 18.7 mmol, Eq: 1.10) was added over 30 min (IPC by GC after derivatization with morpholine, 1 h reaction>99% conversion). After 18 h, the reaction mixture was concentrated to ca 40 mL on the rotavapor at 45° C./90 mbar. The solution was twice diluted with toluene (25 mL) and concentrated to ca 25 mL at 45° C./90 mbar to remove CO₂ and HCl. This solution was added dropwise over 30 min to a mixture of 1 M LiAlH₄ in THF (17.0 mL, 17.0 mmol, Eq: 1.00) and toluene (38 mL) at 0-2° C. The reaction mixture was stirred for 2 h (IPC: SM<0.5%) and slowly added to a stirred mixture of 2 M aqueous HCl (34.0 mL, 67.9 mmol, Eq: 4.00) and heptane (40 mL). The organic phase was separated, washed twice with water (2×40 mL), dried over MgSO₄, filtered and concentrated under reduced pressure to give 7.2 g of the title compound as a crude oil (GC: 99a %). The crude product was dissolved in methanol (140 mL). The solution was polish filtered. The clear colorless filtrate was cooled down to 0°-2° C. and water (14 mL) was added. The solution was seeded. After 2 h, water (28 mL) was added. After a further 4 h at 0° C., the suspension was filtered. The filter cake was washed with a cold (0° C.) mixture of methanol (7 mL) and water (9 mL). The crystals were dried at 40° C./15 mbar to give 6.6 g of the title compound as a white crystalline powder (>99.5a % by GC; 99.5:0.5 e.r.).

### 2-(3,5-dimethoxyphenyl)-2-methylpropanal

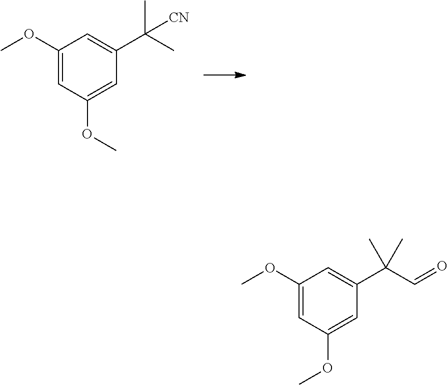

2-(3,5-dimethoxyphenyl)-2-methylpropanenitrile (50 g, 240 mmol, Eq: 1.00) was dissolved in MTBE (250 mL). The solution was cooled to 20° C. and 1 M DIBAH in heptane (288 mL, 288 mmol, Eq: 1.2) was added dropwise over 30 min. After 30 min reaction time, the reaction mixture was poured on a cold (0° C.) stirred mixture of heptane (250 mL) and 2 M aqueous HCl (540 mL, 1.08 mol, Eq: 4.5). After 20 min stirring, the water phase was separated and extracted with heptane (250 mL). The organic phases were washed twice sequentially with 2 M aqueous HCl (250 mL) then with half saturated aqueous NaHCO₃ (250 mL) and half saturated NaCl (250 mL). The organic phase was dried over Na₂SO₄, filtered and concentrated under reduced pressure to give 46.8 g of the title compound as an oil (99.3a % by GC).

### (Z/E)-1,3-dimethoxy-5-(2-methyloct-3-en-2-yl)benzene

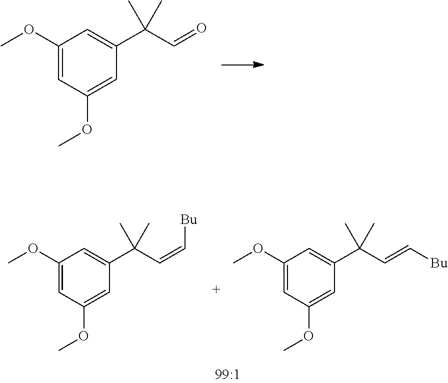

Pentyltriphenylphosphonium bromide (99.5 g, 236 mmol, Eq: 1.1) and 2-(3,5-dimethoxyphenyl)-2-methylpropanal (45 g, 215 mmol, Eq: 1.00) were charged in the reactor, followed by THF (225 mL). The suspension was cooled to 0° C. and a solution of 1 M potassium tert-butoxide solution in THF (236 mL, 236 mmol, Eq: 1.1) was added dropwise over 30 min at 0°−5° C. (turned to a light yellow suspension). After 45 min (IPC by GC: ca 97% conversion) more potassium tert-butoxide solution in THF (17.2 mL, 17.2 mmol, Eq: 0.08) was added and the suspension turned to a deep orange suspension (IPC after 5 min: >99.8% conversion by GC). After 1 h at 0° C., the reaction mixture was poured on a stirred mixture of water (225 mL), heptane (450 mL) and a 70:30 methanol/water solution (450 mL). The aqueous phase was separated and extracted with heptane (225 mL). The organic phases were washed 3 times sequentially with a 70:30 methanol/water solution (300 mL). The organic phases were combined, dried over MgSO₄, filtered and concentrated under reduced pressure to give 55.3 g of the title compound as an oil (98.4% Z isomer+1% E isomer by GC).

### 1,3-dimethoxy-5-(2-methyloctan-2-yl)benzene

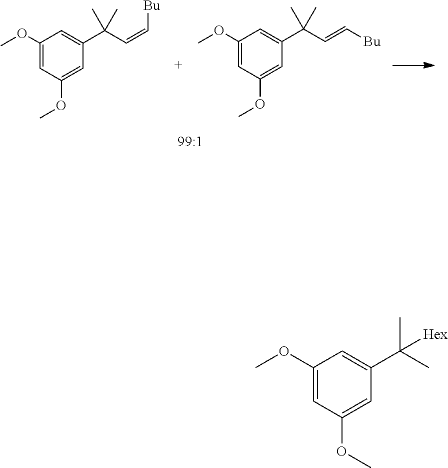

(Z)-1,3-dimethoxy-5-(2-methyloct-3-en-2-yl)benzene (55 g, 210 mmol, Eq: 1.00) was charged in the reactor, followed by AcOEt (869 mL) and 10% Pd/C E101 N/D Evonik (1.13 g, 10.6 mmol, Eq: 0.0505). The Hydrogenation was performed at 30° C. and 4 bar H₂. After 18 h the reaction mixture was filtered and the filtrate was concentrated under reduced pressure to give 53.3 g of the title compound as an oil (98.9a % GC).

### Seed crystals for ((1S,4R)-2-(2,6-dimethoxy-4-(2-methyloctan-2-yl)phenyl)-7,7-dimethylbicyclo[2.2.1]hept-2-en-1-yl)methanol

Seed crystals were obtained from test tube crystallization tests:
From cold heptane (quickly re-dissolved upon warming to RT)
From EtOH/water at RT (first oiled but crystallized overnight)
From MeOH/water at RT

### (1S,4R)-2-(2,6-dimethoxy-4-(2-methyloctan-2-yl)phenyl)-7,7-dimethylbicyclo[2.2.1]hept-2-ene-1-carboxylic Acid Sodium Salt

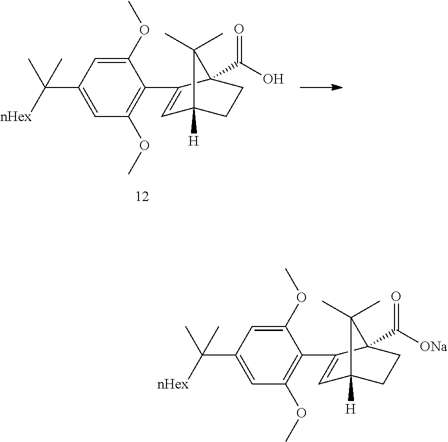

(1S,4R)-2-(2,6-dimethoxy-4-(2-methyloctan-2-yl)phenyl)-7,7-dimethylbicyclo[2.2.1]hept-2-ene-1-carboxylic acid (60 g, 140 mmol) were dissolved in 2-propanol (234 g, 300 mL). 32% aqueous NaOH (16.2 g, 12 mL) was added followed by water (18 mL). The solution was seeded. The resulting suspension was diluted with 2-propanol (100 mL) and water (10 mL). After 1 h at RT, water (40 mL) was added. The resulting solution was cooled to 0-5° C. during which crystallization started. After 1 h, the suspension was filtered. The filter cake was washed with a mixture of cold 0-5° C. 2-propanol/water 9:1 (100 mL) and dried at 50° C./10 mbar to give 42 g of the tile compound.

### (1S,4R)-2-(2,6-dimethoxy-4-(2-methyloctan-2-yl)phenyl)-7,7-dimethylbicyclo[2.2.1]hept-2-ene-1-carboxylic Acid Dicyclohexylamine Salt

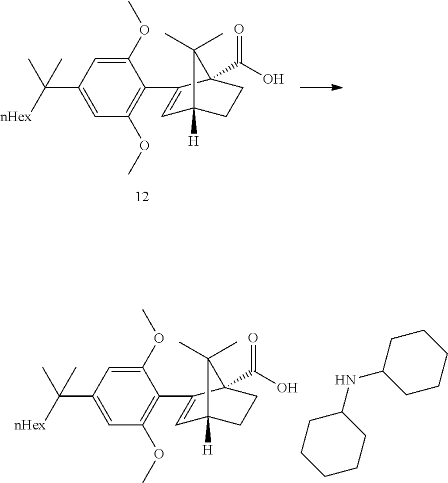

To a stirred solution of (1S,4R)-2-(2,6-dimethoxy-4-(2-methyloctan-2-yl)phenyl)-7,7-dimethylbicyclo[2.2.1]hept-2-ene-1-carboxylic acid (4 g, 9.33 mmol, Eq: 1.00) in heptane (20.0 mL) was added dicyclohexylamine (1.69 g, 1.86 mL, 9.33 mmol, Eq: 1.00). The resulting suspension was stirred at RT for 16 h, then cooled to 0° C. for 1 h. The light yellow suspension was filtered. The filter cake was washed with cold heptane and dried (50° C./10 mbar) to give 3.52 g of the title compound.

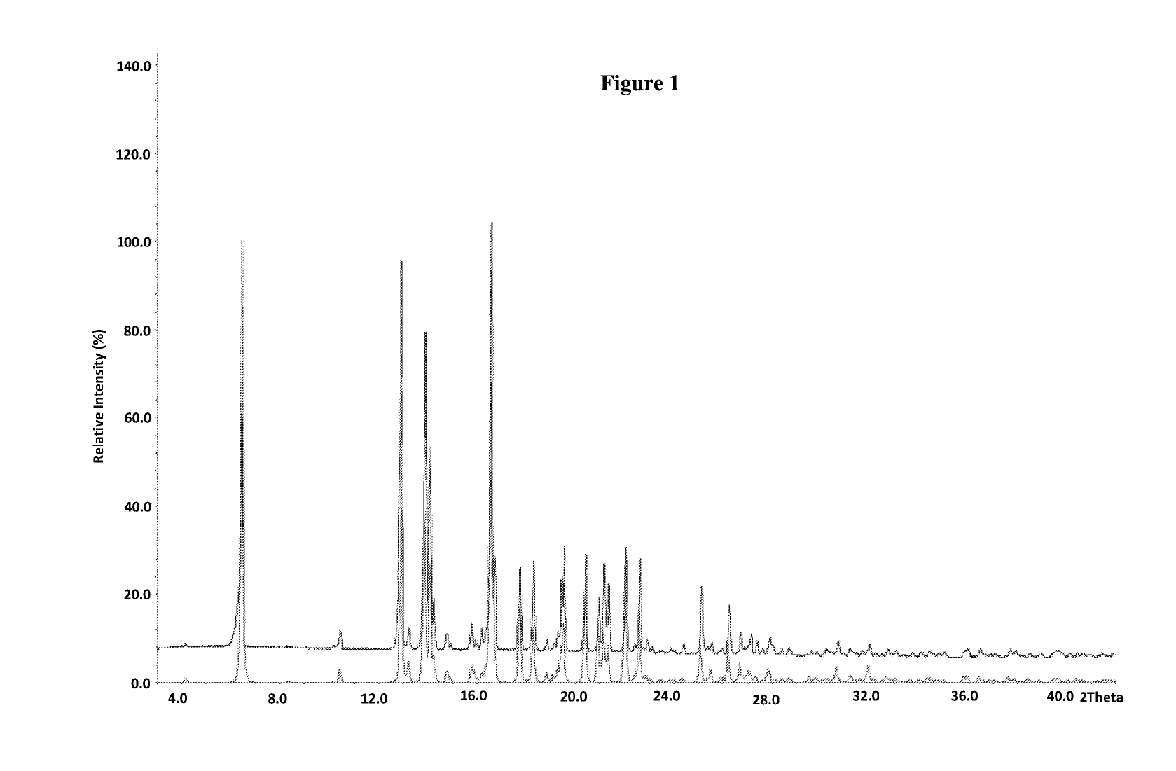

### Crystalline structure of HU-910 ((1S,4R)-2-(2,6-dimethoxy-4-(2-methyloctan-2-yl)phenyl)-7,7-dimethylbicyclo[2.2.1]hept-2-en-1-yl)methanol—FIG. 1

Empirical formula C27 H42 O, Formula weight 414.61

Temperature 89(2) K, Wavelength 0.70000 A

Crystal system, space group Monoclinic, C2

Unit cell dimensions a=33.098(7) A alpha=90 deg. b=7.1740(14)A beta=124.70(3) deg. c=25.105(5) A gamma=90 deg.

Volume 4900.8(17) A \hat{ }3

Z, Calculated density 8, 1.124 Mg/m\hat{ }3

Absorption coefficient 0.071 mm \hat{ }−b 1

F(000) 1824

Crystal size 0.20×0.20×0.03 mm

Theta range for data collection 0.99 to 26.37 deg.

Limiting indices −41<=h<=41, −8<=k<=8, −31<=1<=31

Reflections collected/unique 32019/9870 [R(int)=0.0330]

Completeness to theta=26.37 99.3%

Absorption correction None

Refinement method Full-matrix least-squares on F\hat{ }2

Data/restraints/parameters 9870/1/557

Goodness-of-fit on F\hat{ }2 0.948

Final R indices [I>2sigma(I)] R1=0.0473, wR2=0.1291

R indices (all data) R1=0.0492, wR2=0.1313

Absolute structure parameter 0.9(8)

Largest diff. peak and hole 1.187 and −0.412 e.A\hat{ }−3

1.-23. (canceled)

24. A process for the preparation of HU-910 including the steps of:
(a) Preparation of resorcinol building block comprising: 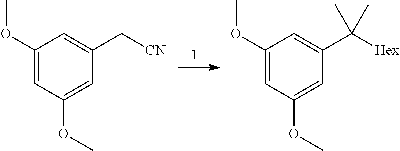
(b) Preparing the triflate ketopinic methyl ester derivative building block comprising: 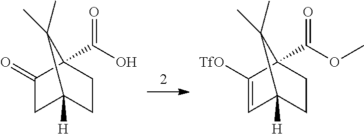 [SMILES: CC1(C)[C@@H]2CC[C@@]1(C(=O)O)C(=O)C2 ; COC(=O)[C@]12CC[C@H](C=C1OS(=O)(=O)C(F)(F)F)C2(C)C]
(c) Coupling the resorcinol building block and the triflate ketopinic methyl ester derivative building block comprising to produce a coupled compound: 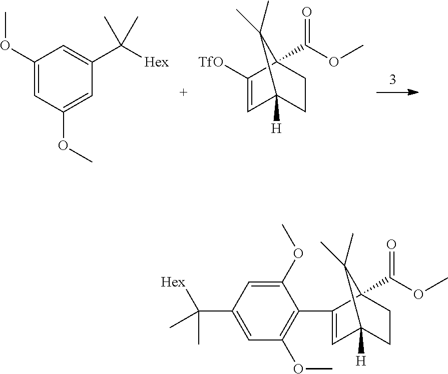
(d) Forming compound HU-910 from said coupled compound:

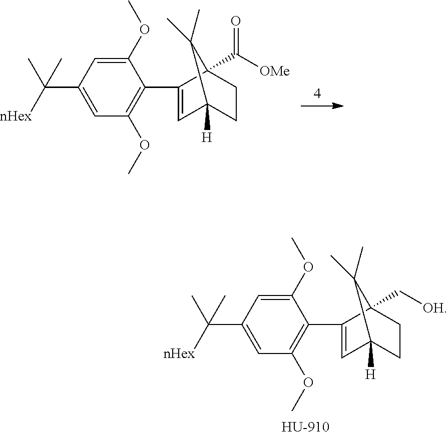

25. The process of claim 24, wherein step 1 comprises the reactions: (1a) dimethylation reaction; (1b) a reduction reaction; (1c) a Wittig reaction and (1d) a hydrogenation reaction.

26. The process of claim 24, wherein step 2 comprises the reactions: (2a) esterification reaction (2b) deprotonation (2c) triflation reaction.

27. The process of claim 24, wherein step 3 comprises the reactions: (3a) a deprotonation of the dimethoxyaryl ring (3b) a reaction of the generated arylmetal with an electrophile (3c) a C—C coupling reaction.

28. The process of claim 24, wherein step 4 comprises a hydrolysis step.

29. The process of claim 24, wherein step 4 comprises the reactions: (4a) a hydrolysis reaction (4b) an acid reduction.

30. The process of claim 24, wherein step 4 comprises the reactions: (4a) a hydrolysis (4b) an acid chloride formation and (4c) a reduction.

31. A crystalline structure of ((1S,4R)-2-(2,6-dimethoxy-4-(2-methyloctan-2-yl)phenyl)-7,7-dimethylbicyclo[2.2.1]hept-2-en- 1-yl)methanol (HU-910) having the following unit cell: a=33.098(7) A alpha=90 deg., b=7.1740(14) A beta=124.70(3) deg. and c=25.105(5) A gamma=90 deg.

32. A crystalline structure of HU-910 according to claim 31, having the following unit cell: a=33.098(7) A alpha=90 deg., b=7.1740(14) A beta=124.70(3) deg. and c=25.105(5) A gamma=90 deg.
